(19) United States
(12) Patent Application Publication
(10) Pub. No.: US 2007/0065378 A1
(43) Pub. Date: Mar. 22, 2007

(54) Sunscreen compositions

(76) Inventors: Brian J. Vondruska, Lyndhurst, OH (US); Laurie J. Marshall, Center Valley, PA (US)

Correspondence Address: LEGAL DEPARTMENT;NOVEON, INC., 9911 BRECKSVILLE ROAD, CLEVELAND, OH, 44141-3247, US

(21) Appl. No.: 11/519,684

(22) Filed: Sep. 12, 2006

Related U.S. Application Data
(60) Provisional application No. 60/718,077, filed on Sep. 16, 2005.

(51) Int. Cl. A61K 8/89; A61K 8/49; A61K 8/37; A61K 8/36

(52) U.S. Cl. 424/59; 424/70.12

(57) ABSTRACT

A composition and method for enhancing the SPF value of UV absorbing agents which are formulated into sunscreen composition and applied to the skin is disclosed. A mixture is formed which includes at least one UV absorbing agent and an effective amount of a dimethicone copolyol wax composition. The composition exhibits a synergistic effect over compositions that contain the UV absorbing agent absent the dimethicone copolyol wax. The mixture is applied to the skin to be protected from excessive exposure to UV radiation.

### RELATED U.S. APPLICATION

This application claims the benefit of priority for U.S. provisional application Ser. No. 60/718,077 filed on Sep. 16, 2005.

### FIELD OF THE INVENTION

The present invention relates to a sunscreen formulation that contains a dimethicone copolyol wax composition prepared by the reaction of a dimethicone copolyol and a high molecular weight natural wax composition selected from beeswax, candelillia, and carnauba wax. The dimethicone copolyol wax composition functions to enhance the Sunscreen Protection Factor (SPF) of selected organic UV absorbing agents. This invention also relates to a method for boosting the SPF value of selected sunscreen active compounds that are topically applied to the skin protecting the skin from excessive exposure to ultraviolet radiation.

### BACKGROUND

Exposure to ultraviolet radiation (UV-A and UV-B hereafter UV or ultraviolet radiation) from sunlight is a major concern as more people pursue work and leisure activities outdoors. In addition, the growing popularity of artificial tanning beds and booths has exposed an increasing number of the public to the harmful effects of ultraviolet light. The short and long term hazards of prolonged exposure to ultraviolet radiation on the skin are well documented. The major short term effect is the sun burning and/or blistering of the skin, medically referred to as “erythema”. Long term adverse effects include the premature aging of the skin characterized by wrinkling, yellowing, cracking and loss of elasticity (sagging). A potentially fatal long term effect associated with prolonged exposure to ultraviolet radiation is malignant transformations of the skin's surface. Various epidemiological studies have linked prolonged sun exposure to implications of skin cancer.

These effects are taken very seriously by the public as indicated by the considerable growth in the sunscreen products market. Sunscreens are cosmetic compositions which are topically applied to the skin to provide protection against the adverse effects of ultraviolet radiation. They are primarily formulated as tanning and sunscreen lotions. Conventional sunscreens are prepared using cosmetically acceptable lotions, oils, creams, gels, and emulsions (oil-in-water and water-in-oil). Sunscreen compositions typically contain organic UV absorbing agents which protect the skin by absorbing the harmful wavelengths of ultraviolet radiation before reaching the surface to penetrate the skin.

The effectiveness of UV protection afforded by a sunscreen composition is expressed as the SPF value. The SPF value measures the amount of protection from the sun provided before a certain level of erythema is experienced. The SPF value is derived from the minimal erythemal dose (MED). The MED is defined as the least exposure dose at a specified wavelength that will elicit a delayed erythematic response. The MED indicates the amount of energy reaching the skin and the responsiveness of the skin to the radiation. The absolute dose differs from person to person and is largely dependent on one's genetic predisposition and ethnic origin. The SPF of a particular UV absorbing agent is obtained by dividing the MED of protected skin by the MED of unprotected skin. The SPF value is an indication of how many times longer a person can stay in the sun with use of a sunscreen (compared to the same person with unprotected skin) before that person will experience 1 MED. For example, utilizing a sunscreen with an SPF value of 6 will allow an individual to stay in the sun six times longer before receiving the MED. The higher the SPF value of a particular UV absorber, the more effective that agent is in preventing sunburn. Public awareness of the problems of exposure to sunlight has led to a demand for sunscreen products with high SPF values (greater than 8).

To achieve higher SPF values, generally, greater quantities of the UV absorbing agent or a combination of UV absorbing agents are formulated into a sunscreen composition. However, increasing the quantities of UV absorbing agents and/or employing a combination of various UV absorbing agents into a sunscreen composition present certain challenges in formulation. Formulating personal care products such as tanning and sunscreen lotions is becoming increasingly complex as efforts are made to promote water and perspiration resistance, spreadability, reduction in tackiness, stability, compatibility, feel, and efficacy of the final product. Furthermore, concerns have arisen regarding the potential for irritation resulting from organic UV absorbing agents penetrating into a user's skin. Consequently, there typically exists a regulatory limit on the concentration and frequency of use for many organic UV absorbers.

Providing a topical sunscreen composition with a high SPF value is difficult to achieve without the negative characteristics associated with using larger quantities of organic UV absorbing agents. Accordingly, there is a need for a cosmetically acceptable sunscreen composition having a high SPF value which will protect the skin from the adverse effects of prolonged exposure to UV radiation, without having to increase the quantity of organic UV absorbing agent in the composition.

### DESCRIPTION OF EXEMPLARY EMBODIMENTS

The present invention relates to a topical sunscreen composition that has an enhanced photoprotective effect. It has been unexpectedly discovered that the SPF value of a sunscreen composition can be enhanced or boosted when a dimethicone copolyol wax is combined with at least one organic UV absorbing agent selected from 2-ethylhexyl 4-methoxycinnamate (octyl methoxycinnamate), 2-phenylbenzimidazole-5-sulfonic acid (phenylbenzimidazole sulfonic acid), (2-hydroxy-4-methoxyphenyl)phenylmethanone (benzophenone-3), 2-ethylhexyl salicylate (octyl salicylate), 2-propanoic acid, 2-cyano-3-diphenyl, 2-ethylhexyl ester (2-ethylhexyl-2-cyano-3,3-diphenylacrylate), and 2-ethylhexyl 4-(dimethylamino)benzoate (Padimate O). The foregoing UV absorbing agents are well known and are commercially available.

In addition to the organic UV absorbing agents set forth above, the compositions of the present invention can optionally comprise at least one inorganic UV absorbing agents. Examples of inorganic UV absorbing agents include, but are not limited to zinc oxide, titanium dioxide, calamine, and mixtures thereof.

A safe and effective amount of UV absorbing agent is used in the sunscreen compositions of the present invention. By “safe and effective” is meant an amount sufficient to provide the skin with protection against UV radiation without concomitant side effects or adverse skin reactions. The amount of organic UV absorber employed in the sunscreen composition can range from about 0.5 wt. % to about 30 wt. % in one aspect, from about 1 wt. % to about 25 wt. % in another aspect, and from about 2 wt. % to about 15 wt. % in a further aspect of the invention (based upon the weight of the total sunscreen composition). Exact amounts will vary depending on the UV absorbing agent(s) chosen and the desired SPF value.

If an optional inorganic UV absorbing agent(s) is utilized as an auxiliary UV absorber, the amount can range from about 0.5 wt. % to about 15 wt. 5 in one aspect, from about 1 wt. % to about 8 wt. % in another aspect, and from about 3 wt. % to about 5 wt. % in still another aspect of the invention (based on the total weight of the sunscreen composition).

The dimethicone copolyol wax component of the invention is utilized in an amount ranging from about 0.1 wt. % to about 20 wt. % in one aspect, from about 0.5 wt. % to about 10 wt. % in another aspect, and from about 0.5 wt. % to about 5 wt. % in a still further aspect of the invention (based upon the weight of the total sunscreen composition).

This invention also relates to a method for boosting the SPF value of selected sunscreen active compounds that are topically applied to the skin. The method includes the steps of forming a composition which includes at least one of the organic UV absorbing agents enumerated above and an effective amount of a dimethicone copolyol wax and applying the composition to the surface of the skin to be protected from exposure to UV radiation. When the dimethicone copolyol wax is mixed with at least one of the organic UV absorbing agents, the composition exhibits an increased SPF value than would be exhibited by the UV absorbing agent(s) in the absence of the dimethicone copolyol wax. In accordance with the present invention it is possible to achieve increases in SPF value in excess of 50 percent and upwards. This is of significant advantage in that it is possible to achieve increased skin protection primarily without adding larger quantities of the UV absorbing agent(s) which often is potentially irritating to skin tissue and deleterious to multi-component personal care formulations. Therefore, the stability and aesthetic challenges experienced with larger quantities of organic and inorganic sunscreen agents can be avoided. The compositions and methods of the present invention feel comfortable on the skin, look more appealing are water resistant and achieve an increased SPF value.

In one embodiment of the invention the dimethicone copolyol wax composition comprises a dimethicone copolyol wax component selected from a polymer conforming to the formula (I): 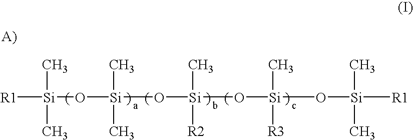
wherein:
a is an integer ranging from 0 or 1 to 2000;
b is an integer ranging from 1 to 20; and
c is an integer ranging from 0 to 20, subject to the proviso that a, b, and c can not all be zero at the same time;
R1 can be the same or different and is —CH₃, or —(CH₂)₃—O—(CH₂CH₂O)ₓ—(CH₂CH(CH₃)O)_{y}—(CH₂CH₂O)ₙ—C(O)R;
R2 is —CH₃, or —(CH₂)₃—O—(CH₂CH₂O)ₓ—(CH₂CH(CH₃)O)_{y}—(CH₂CH₂O)ₙ—C(O)R;
Each of x, y, and z in R1 and R2 above is an integer ranging from 0 to 20 in one aspect, x is 1 to 5, y is 0, and z is 0 in another aspect, and x is 1, y is 0 and z is 0 in a further aspect, with the proviso that R1 and R2 can not be —CH₃ at the same time, and x, y, and z can not be 0 at the same time;
R3 is —(CH₂)ₙCH₃, with n being an integer from 1 to 17; and
R is a hydrocarbon group containing from about 19 to about 52 carbon atoms in one aspect, from about 20 to about 38 carbon atoms in another aspect and from 21 to about 35 carbon atoms in a further aspect of the invention. In another embodiment R is an alkyl group of from about 19 to about 52 carbon atoms in one aspect, from about 20 to about 38 carbon atoms in another aspect and from 21 to about 35 carbon atoms in a further aspect of the invention; and

B) an optional alcohol conforming to the formula:
R6-OH
Wherein R6 is an alkyl group having from about 20 to about 38 carbon atoms.

In another embodiment of the invention the dimethicone copolyol wax component (A) conforms to the formula (II): 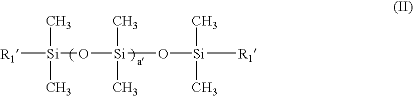
wherein:
a′ is an integer from 1 to 2000;
R₁′ is —(CH₂)₃—O—(CH₂CH₂O)ₙ—(CH₂CH(CH₃)O)_{y}—(CH₂CH₂O)_{z}—C(O)R, with each of x, y, and z representing an integer from 0 to 5, subject to the proviso that x, y, and z can not be zero at the same time and the sum of x, y, and z together can not be greater than 5; and
R is a hydrocarbon group containing from about 19 to about 52 carbon atoms in one aspect, from about 20 to about 38 carbon atoms in another aspect and from 21 to about 35 carbon atoms in a further aspect of the invention. In another embodiment R is an alkyl group of from about 19 to about 52 carbon atoms in one aspect, from about 20 to about 38 carbon atoms in another aspect and from 21 to about 35 carbon atoms in a further aspect of the invention.

The dimethicone copolyol wax compositions of the present invention are known in the art and are prepared by the esterification and/or transesterification reaction of a dimethicone copolyol and a high molecular weight natural wax composition selected from beeswax, candelillia, and carnauba wax. The starting materials and the methods of preparation of the copolyol waxes of the present invention are described in U.S. Pat. No. 5,733,533 which is incorporated herein by reference. The dimethicone copolyol starting materials are disclosed in U.S. Pat. Nos. 5,136,063 and 5,180,843, the disclosures of which are incorporated herein by reference. The high molecular weight natural waxes used in the esterification reaction are commercially available from J. W. Hanson Company Inc., Woodbury, N.Y. and Well, Naturally Products Ltd., 12706-114A Avenue, Surrey, British Columbia, Canada.

The compounds of the present invention are prepared by the esterification and transesterification reaction of the wax and the dimethicone copolyol. In an exemplary embodiment, the reaction is carried out with a molar ratio of 0.5:1 to 1:0.5 ratio of carboxy and/or ester groups in the wax to hydroxy groups in the copolyol. In another aspect, the molar ratio of wax to copolyol is 1:1. The wax and the copolyol are added to a suitable reaction vessel under agitation. The reactants are heated to 160-250° C. in one aspect and between 180-200° C. in another aspect. Optionally, an esterification catalyst selected from p-toluene sulfonic acid, tin oxylate, sulfuric acid, and other esterification catalysts can be employed in the reaction. The reaction is conducted for three to eight hours. An alkyl alcohol R6-OH is generated as a by-product of the transesterification reaction. Its presence is beneficial as a formulation aid when the dimethicone copolyol wax is formulated into personal care compositions, including sunscreen compositions.

In one embodiment the natural wax reactant is selected from beeswax (CAS No. 8012-89-3). The chemical composition of beeswax is complex and can vary slightly depending on the specie of bee producing the wax. Beeswax contains a variety of compounds including long alkyl chain acids and esters that are idyllic for esterification and transesterification reactions with the dimethicone copolyol. This composition distinguishes the material as a wax rather than a fat because it is composed of long chain carbon components including alkanes containing 21 to 33 carbon atoms, free alkyl acids containing 22 to 30 carbon atoms (e.g., cerotic acid), and esters that contain 40 to 52 carbon atoms (Kameda T. 2004, Molecular Structure of Crude Beeswax Studied by Solid-State ¹³C NMR, Journal of Insect Science, 4:29). Triglycerides and diglycerides containing lower carbon chain (<C19) fatty acid moieties, typical of fats, are absent.

In one embodiment the dimethicone copolyol wax conforms to the following formula (III): 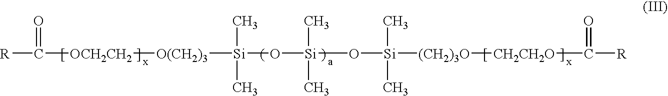
wherein:
a is an integer from 1 to 2000;
x independently represents an integer selected from 1, 2, 3, 4, and 5; and the terminal acyl moieties RC(O)— can be the same or different and represent residues of beeswax fatty acids and esters containing 19 to 52 carbon atoms.
The compounds of formula (III) can be obtained by the esterification and/or transesterification reaction of a dimethicone copolyol represented by the formula (IV): 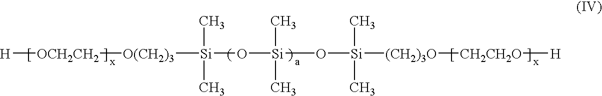
wherein a and x are as defined immediately above with the fatty acids and esters contained in beeswax.

In another embodiment the R group set forth in formula (III) represents an alkyl group obtained by the esterification and/or transesterification reaction of the dimethicone copolyol represented by formula (IV) and alkyl groups contained in beeswax fatty acids and esters wherein said alkyl groups contain 19 to 52 carbon atoms in one aspect, 20 to 40 carbon atoms in another aspect, and 22 to 30 carbon atoms in a further aspect of the invention.

Dimethicone copolyol beeswax compositions are commercially marketed under the Ultrabee® 25 trademark by Noveon, Inc., Cleveland, Ohio, and are described in Noveon Technical Data Sheet TDS-351, Jul. 18, 2005.

The sunscreen compositions of the present invention comprise the specified UV absorbing agent(s) in combination with a dimethicone copolyol wax in pharmaceutically-acceptable sunscreen carrier materials and optional components selected as appropriate for the form and characteristics desired for a particular composition. The sunscreen compositions may be in the form of creams, gels, lotions, and oils. The term “pharmaceutically-acceptable sunscreen carrier”, as used herein, means one or more substantially non-irritating compatible diluents which are suitable for topical application to the skin. The term “compatible”, as used herein, means that the components of the carrier must be capable of being formulated with the UV absorbing agent, the dimethicone copolyol wax, and with other ingredients typically contained in sunscreen and personal care formulations in a manner such that there is no interaction which would substantially reduce the efficacy of the composition during use for protecting the skin from the effects of damaging UV radiation.

Suitable carrier materials useful for sunscreen compositions are well known in the art and their selection are readily made by one of ordinary skill in the art. Exemplary of some of the many ingredients which may be selected as suitable carrier materials include but are not limited to, water, natural and synthetic oils, lower alcohols, and mixtures thereof. Exemplary lower alcohols being selected from ethanol, isopropyl alcohol, propylene glycol, glycerol, and sorbitol. Exemplary natural and synthetic oils can be selected from any of the oils set forth in the disclosure below.

The sunscreen composition of the invention may be formulated as water-in oil or oil-in-water dispersions, oils or oil/alcohol lotion, a vesicular dispersion of an ionic or nonionic amphiphilic lipid, a gel, a solid stick, or an aerosol formulation.

When formulated as water-in oil or oil-in-water dispersions, in one aspect, the pharmaceutically acceptable carrier can comprise 5 to 50% of an oil phase, 0.5 to 20% of an emulsifier and 30 to 90% of water, each by weight based on the total weight of the carrier. The oil phase can comprise any oil conventionally used in cosmetic formulations, an emollient, e.g., one or more of a fatty alcohol; hydrocarbon oil; a natural or synthetic triglyceride; a wax including esters of long-chain acids and alcohols as well as compounds having wax-like properties; a silicone oil; a fatty acid ester or a fatty alcohol; and lanoline-containing products.

Examples of fatty alcohols include but are not limited to cetyl alcohol, stearyl alcohol, octyldodecanol, cetearyl alcohol, and oleyl alcohol.

Examples of hydrocarbon oils include but are not limited to mineral oil (light or heavy), petrolatum (yellow or white), polyethylene, paraffin, squalane, microcrystalline wax, ceresin, polybutene, and hydrogenated polyisobutene.

Examples of a natural or synthetic triglycerides include but are not limited to castor oil, caprylic/capric triglyceride, hydrogenated vegetable oil, sweet almond oil, wheat germ oil, sesame oil, hydrogenated cottonseed oil, coconut oil, wheat germ glycerides, avocado oil, corn oil, trilaurin, hydrogenated castor oil, shea butter, cocoa butter, soybean oil, mink oil, sunflower oil, safflower oil, macadamia nut oil, olive oil, hydrogenated tallow, apricot kernel oil, hazelnut oil. and borage oil.

Examples of a wax including esters of long-chain acids and alcohols as well as compounds having wax-like properties include but are not limited to carnauba wax, beeswax (white or yellow), lanolin, candelillia wax, ozokerite, lanolin oil, paraffin, Japan wax, microcrystalline wax, ceresin, jojoba oil, cetearyl esters wax, synthetic jojoba oil, synthetic beeswax, and lanolin wax.

Examples of silicone oil include but are not limited to dimethicone and cyclomethicone oils.

Examples of a fatty acid ester or a fatty alcohol include isopropyl myristate, isopropyl palmitate, octyl palmitate, isopropyl lanolate, acetylated lanolin alcohol, the benzoate of C12 to C15 alcohols, cetearyl octanoate, cetyl palmitate, myristyl myristate, myristyl lactate, cetyl acetate, propylene glycol dicaprylate/caprate, decyl oleate, acetylated lanolin, stearyl heptanoate, diisostearyl malate, octyl hydroxystearate, octyl hydroxystearate, and isopropyl isostearate.

Examples of lanoline-containing products include but are not limited to lanolin, lanolin oil, isopropyl lanolate, acetylated lanolin alcohol, acetylated lanolin, hydroxylated lanolin, hydrogenated lanolin, and lanolin wax.

The emulsifier may comprise any emulsifier conventionally used in cosmetic formulations. Suitable emulsifiers include anionic, cationic, amphoteric, zwitterionic, and nonionic surfactants of various HLB values, molecular weight, polarity, and solubility. For esthetic, safety reasons and due to their mildness to human skin nonionic emulsifiers are preferred. A number of these emulsifiers are well known in the trade and are listed in the “McCutcheon's Emulsifiers and Detergents”, 2004 Edition.

Exemplary emulsifier classes include acyl lactylates, alkyl phosphates, carboxylic acid copolymers, esters and ethers of glucose, esters of glycerin, esters of propylene glycol, esters of sorbitan anhydrides, esters of sorbitol, ethoxylated ethers, ethoxylated alcohols, fatty acid amides, fatty acid esters of polyethylene glycol, fatty esters of polypropylene glycol, polyoxyethylene fatty ether phosphates, soaps. and mixtures thereof. Emulsifiers can include, but are not limited to, ceteareth-20, ceteth-10, cetyl phosphate, diethanolamine cetyl phosphate, glyceryl stearate, PEG-100 stearate, polyethylene glycol 20 sorbitan monolaurate, polyethylene glycol 5 soya sterol, polysorbate 60, polysorbate 80, potassium cetyl phosphate, PPG-2 methyl glucose ether distearate, PPG-2 isoceteth-20, steareth-20, and mixtures thereof.

In addition to the carrier materials to aid in distribution of the UV absorbing agent onto the skin, some embodiments may include optional additives to improve the cosmetic properties of the sunscreen composition. These optional additives can comprise one or more of a wide variety of components well known to those skilled in the art, such as chelators, fragrances, humectants/humectant skin conditions, lubricants, emollients, neutralizers, preservatives, auxiliary solvents, spreading aids, film forming polymers, viscosity modifiers/emulsifiers, and the like, and combinations thereof, as well as any other compatible ingredient usually employed in cosmetics. Exemplary skin care compositions utilizing such components include those of U.S. Pat. Nos. 5,073,372, 5,380,528, 5,599,549, 5,874,095, 5,883,085, 6,013,271, and 5,948,416, all incorporated herein by reference. Such components are also described in detail in well known references such as Mitchell C. Schlossman, The Chemistry and Manufacture of Cosmetics, Volumes I and II, Allured Publishing Corporation, 2000. It will be appreciated by the skilled artisan that many of the components of personal care compositions can serve dual or multipurpose roles and generally can be added to the composition at any stage of the formulation process.

Suitable chelators include EDTA (ethylene diamine tetraacetic acid) and salts thereof such as disodium EDTA, citric acid and salts thereof, cyclodextrins, and the like, and mixtures thereof. Such suitable chelators typically comprise about 0.001 wt. % to about 3 wt. % in one aspect, from about 0.01 wt. % to about 2 wt. % in a further aspect, and from about 0.01 wt. % to about 1 wt. % in a still further aspect of the invention (based on the total weight of the sunscreen composition).

Suitable humectant skin conditioners include allantoin; pyrrolidonecarboxylic acid and its salts; hyaluronic acid and its salts; sorbic acid and its salts; urea; lysine, arginine, cystine, guanidine, and other amino acids; polyhydroxy alcohols such as glycerin, propylene glycol, hexylene glycol, hexanetriol, ethoxydiglycol, dimethicone copolyol, and sorbitol, and the esters thereof; polyethylene glycol; glycolic acid and glycolate salts (e.g. ammonium and quaternary alkyl ammonium); lactic acid and lactate salts (e.g. ammonium and quaternary alkyl ammonium); sugars and starches; sugar and starch derivatives (e.g. alkoxylated glucose); D-panthenol; lactamide monoethanolamine; acetamide monoethanolamine; and the like, and mixtures thereof. Preferred humectants include the C₃ to C₆ diols and triols, such as glycerin, propylene glycol, hexylene glycol, hexanetriol, and the like, and mixtures thereof. Such suitable humectants typically comprise about 1 wt. % to about 10 wt. % in one aspect, from about 2 wt. % to about 8 wt. % in another aspect, and from about 3 wt. % to about 5 wt. % in still another aspect of the invention (based on the total weight of the sunscreen composition).

Suitable lubricants include volatile silicones, such as cyclic or linear polydimethylsiloxanes, and the like. The number of silicon atoms in cyclic silicones preferably is from about 3 to about 7 and more preferably 4 or 5. Exemplary volatile silicones, both cyclic and linear, are available from Dow Corning Corporation as Dow Corning 344, 345, and 200 fluids; Union Carbide as Silicone 7202 and Silicone 7158; and Stauffer Chemical as SWS-03314.

The linear volatile silicones typically have viscosities of less than about 5 cP at 25° C., while the cyclic volatile silicones typically have viscosities of less than about 10 cP at 25° C. “Volatile” means that the silicone has a measurable vapor pressure. A description of volatile silicones can be found in Todd and Byers, “Volatile Silicone Fluids for Cosmetics”, Cosmetics and Toiletries, Vol. 91, January 1976, pp. 27 to 32, incorporated herein by reference. Other suitable lubricants include polydimethylsiloxane gums, aminosilicones, phenylsilicones, polydimethyl siloxane, polydiethylsiloxane, polymethylphenylsiloxane, polydimethylsiloxane gums, polyphenyl methyl siloxane gums, amodimethicone, trimethylsiloxyamodimethicone, diphenyl-dimethyl polysiloxane gums, and the like. Mixtures of lubricants can also be used. Such suitable lubricants typically comprise from about 0.10 wt. % to about 15 wt. %, from about 0.1 wt. % to about 10 wt. %, and from about 0.5 wt. % to about 5 wt. % of the total weight of the sunscreen composition according to various aspects of the invention.

Suitable emollients include mineral oil; stearic acid; propylene glycol isoceteth-3 acetate, fatty alcohols such as cetyl alcohol, cetearyl alcohol, myristyl alcohol, behenyl alcohol, and lauryl alcohol; cetyl acetate in acetylated lanolin alcohol, isostearyl benzoate, dicaprylyl maleate, caprylic, and capric triglyceride; petrolatum, lanolin, coco butter, shea butter, beeswax, and esters thereof; ethoxylated fatty alcohol esters such as ceteareth-20, oleth-5, and ceteth-5; avocado oil or glycerides; sesame oil or glycerides; safflower oil or glycerides; sunflower oil or glycerides; botanical seed oils; volatile silicone oils; non-volatile emollients, and the like, and mixtures thereof. Suitable non-volatile emollients include fatty acid and fatty alcohol esters, highly branched hydrocarbons, and the like, and mixtures thereof. Such fatty acid and fatty alcohol esters include decyl oleate, butyl stearate, myristyl myristate, octyldodecyl stearoylstearate, octylhydroxystearate, di-isopropyl adipate, isopropyl myristate, isopropyl palmitate, ethyl hexyl palmitate, isodecyl neopentanoate, octyldodecy neopentanoate, C₁₂ to C₁₅ alkyl benzoate, diethyl hexyl maleate, PPG-14 butyl ether and PPG-2 myristyl ether propionate, cetearyl octanoate, cetyl ethylhexanoate, and the like, and mixtures thereof. Suitable highly branched hydrocarbons include isohexadecane and the like, and mixtures thereof. In one aspect of the invention moisture barriers and/or emollients, alone or in combination, typically comprise from about 1 wt. % to about 20 wt. % based on the weight of the sunscreen composition, from about 2 wt. % to about 15 wt. % in another aspect, and from about 3 wt. % to about 10 wt. % in still another aspect.

Suitable neutralizers include triethanolamine, aminomethyl propanol, ammonium hydroxide, potassium hydroxide, sodium hydroxide, other alkali hydroxides, borates, phosphates, pyrophosphates, cocamine, oleamine, diisopropanolamine, diisopropylamine, dodecylamine, PEG-15 cocamine, morpholine, tetrakis(hydroxypropyl)ethylenediamine, triamylamine, triethanolamine, triethylamine, tromethamine (2-Amino-2-Hydroxymethyl-1,3-propanediol, and the like, and mixtures thereof. Such suitable neutralizers typically comprise from about 0.01 wt. % to about 3 wt. % in one aspect, and from about 0.1 wt. % to about 1 wt. % in another aspect of the invention (based on the total weight of the sunscreen composition.

Suitable preservatives include polymethoxy bicyclic oxazolidine, methylparaben, propylparaben, ethylparaben, butylparaben, benzoic acid and the salts of benzoic acid, benzyltriazole, DMDM hydantoin (also known as 1,3-dimethyl-5,5-dimethyl hydantoin), imidazolidinyl urea, diazolidinyl urea, phenoxyethanol, phenoxyethylparaben, methylisothiazolinone, methylchloroisothiazolinone, benzoisothiazolinone, triclosan, sorbic acid, salicylic acid salts, and the like, and mixtures thereof. Such suitable preservatives typically comprise from about 0.01 wt. % to about 1.5 wt. %, from about 0.1 wt. % to about 1 wt. %, and from about 0.3 wt. % to about 1 wt. % of the total weight of the sunscreen composition according to various aspects of the present invention.

Suitable auxiliary solvents include lower monoalcohols such as, for example, C₁ to C₅ monoalcohols. Representative lower monoalcohols include but are not limited to methanol, ethanol, propanol, and isopropyl alcohol.

Suitable spreading aids include hydroxypropyl methylcellulose, hydrophobically modified cellulosics, xanthan gum, cassia gum, guar gum, locust bean gum, dimethicone copolyols of various degrees of alkoxylation, boron nitride, talc, and the like, and mixtures thereof. In one aspect of the invention spreading aids typically comprise from about 0.01 wt. % to about 5 wt. %, from about 0.1 wt. % to about 3 wt. % in a further aspect, and from about 0.1 wt. % to about 2.0 wt. % in a further aspect (based on the total weight of the sunscreen composition).

A suitable film former includes copolymer of eicosene and vinyl pyrrolidone marketed under the Ganex® trademark by GAF Chemical Corporation. The film former typically comprises about 0.1 wt. % to about 20 wt. % in one aspect, from about 0.3 wt. % to about 5 wt. % in another aspect, and from about 0.5 wt. % to about 3 wt. % in a further aspect of the invention (based on the total weight of the sunscreen composition).

Suitable viscosity modifiers/emulsifiers include natural, semi-synthetic, and synthetic polymers. Examples of natural and modified natural polymers include cassia, modified cassia (e.g., 2-hydroxy-3-(trimethylammonium)propyl cassia galactomannan chloride, hydroxypropyl cassia galactomannan), guar, cationically modified guar (e.g., guar hydroxypropyltrimonium chloride), and xanthan gums, cellulosics, modified cellulosics (e.g., carboxymethyl cellulose, hydroxymethyl cellulose, hydroxyethyl cellulose), starches, polysaccharides, and the like. Examples of synthetic polymers include crosslinked homopolymers of (meth)acrylic acid and crosslinked copolymers of (meth)acrylic acid with C₁ to C₃₀ alkyl esters of (meth)acrylic acid, available from Noveon, Inc. and marketed under the Carbopol® trademark (e.g., grade designations 934, 954, 980, 1342, 1353, and Aqua SF-1), hydrophobically modified vinyl or acrylate copolymers, and hydrophobically modified non-ionic polyurethane polymers, and the like. A commercially available hydrophobically modified acrylate copolymer is marketed by Noveon, Inc. under the Pemulen® trademark (e.g., grade designations TR-1 and TR-2). Mixtures can also be used. In one aspect of the invention viscosity modifiers/emulsifiers, alone or in combination, typically comprise from about 0.1 wt. % to about 5 wt. %, from about 0.3 wt. % to about 3 wt. % in another aspect, and from about 0.5 wt. % to about 2 wt. % in a further aspect based on the total weight of the sunscreen composition.

The sunscreen compositions of the present invention comprise from about 50 wt. % to about 99 wt. % pharmaceutically acceptable carrier material (which is typically one or more of the carrier ingredients enumerated above) based on the weight of the total components in the composition.

Several overlapping ranges of ingredient weight percentages have been disclosed. It will be recognized and understood by one of ordinary skill in the art that when formulating the compositions of the present invention the amount of a particular component or ingredient will be selected from the disclosed range according to the properties desired for the composition and that the sum of the amounts of the individual components or ingredients present in a particular composition can not exceed 100 percent.

The following examples further describe and demonstrate embodiments within the scope of the present invention. These examples are presented solely for the purpose of illustration and are not to be construed as limitations of the present invention since many variations thereof are possible without departing from the spirit and scope thereof.

### EXAMPLES

### Sunscreen Formulations

Various experimental sunscreen formulations containing selected UV absorbing agents and the dimethicone copolyol waxes of the invention were formulated with the ingredients set forth in Tables 1 and 2 below.

All of the sunscreen compositions except those that contained 2-phenylbenzimidazole-5-sulfonic acid (PBSA) as the sole UV absorbing agent or in combination with other UV absorbing agents were formulated with the ingredients set forth in Table 1 according to the following method.

PART A Component

1. Disodium EDTA was dissolved in deionized water and heated to approximately 50° C.
2. Carbopol® 980 polymer was then dispersed in the disodium EDTA water solution and mixed for approximately 15 minutes.
3. Pemulen® TR-2 polymer was added to the solution and dispersed by mixing for approximately 15 minutes.
4. Propylene glycol was added to the solution and heated to approximately 75° C. with mixing.
   PART B Component
5. Into a separate vessel were placed the Part B components enumerated in Table 1. The components were heated to approximately 75° C. with mixing. The Part B Components were then added to the Part A components.
   Part C Component
6. The pH of the combined Part A+B composition was adjusted to 5.65 using triethanolamine (TEA 99%).
   Part D Component
7. The temperature of the Part C component mixture was cooled with mixing until the temperature reached approximately 45° C. To the Part C mixture was then added the Part D component.
8. The sunscreen formulation was cooled to ambient room temperature.

Sunscreen compositions containing 2-phenylbenzimidazole-5-sulfonic acid (PBSA) or a combination of PBSA with a secondary UV absorber were formulated with the ingredients set forth in Table 2 as according to the following method.

Part A Component

1. Carbopol® 980 polymer was dispersed in deionized water and mixed for approximately 15 minutes.
2. Pemulen® TR-2 polymer was then dispersed in the water solution and mixed for approximately 15 minutes.
3. Propylene glycol was added to the water solution which was then heated to approximately 75° C. under mixing.
4. The pH of the composition was adjusted 6.5 using triethanolamine (TEA 99%).
   Part B Component
5. Into a separate vessel was dissolved disodium EDTA in warm water heated to approximately 50° C.
6. Into a separate vessel the Part B components enumerated in Table 2 were placed with mixing. The pH of the composition was adjusted to 7.5 using triethanolamine (TEA 99%).
7. The Part B composition was added to the Part A composition under mixing and the combined A+B composition was heated to approximately 75° C.
   Part C Component
8. Into a separate vessel was added the Part C components set forth in Table 2. The components were blended with mixing and then heated to approximately 75° C. under mixing. The Part C composition was then blended with the combined Part A+B composition.
   PART D Component
9. The pH of the A+C+D composition was adjusted to 7.5 using triethanolamine (TEA 99%).
   PART E Component
10. The temperature of the composition obtained in Part D was cooled with mixing until the temperature reached approximately 45° C. To the Part D mixture was then added the Part E component.

11. The sunscreen formulation was cooled to ambient room temperature.

TABLE 1

| Ingredient/ | | | Trade Name |
|---|---|---|---|
| INCI-CTFA Name | Wt. % | Function | (Supplier) |
| PART A | | | |
| 1. Deionized Water | q.s. to 100 | | |
| 2. Carbomer | 0.25 | Rheology | Carbopol ® 980 |
| | | Modifier | polymer (Noveon) |
| 3. C10-30 Alkyl | 0.20 | Rheology | Pemulen ® TR-2 |
| Acrylates | | Modifier | polymer (Noveon) |
| Crosspolymer | | | |
| 4. Propylene Glycol | 2.00 | Humectant | |
| 5. Disodium ETDA | 0.10 | Chelating | Versene ™ NA (Dow) |
| | | Agent | |
| PART B | | | |
| 6. UV Absorber(s) | See | UV Absorber | |
| | Examples | | |
| 7. Bis- | See | SPF Booster | Ultrabee ® 25 silicone |
| hydoxyethoxypropyl | Examples | | (Noveon) |
| Dimethicone beeswax | | | |
| esters | | | |
| 8. Cetearyl Alcohol and | See Note | Emulsifier | Promulgen D |
| Ceteareth-20 | Below | | (Chemron) |
| 9. Stearic Acid | See Note | Emulsifier | Hystrene 5016 NF |
| | Below | | (CK Witco) |
| 10. C12-15 Alkyl | 0.00 or 5.00 | Emollient | Finsolv ® TN (Finetex) |
| Benzoate | | | |
| PART C | | | |
| 11. Triethanolamine | 0.75, q.s. to | Neutralizer | |
| (99%) | pH 5.65 | | |
| PART D | | | |
| 12. Propylene Glycol | 1.00 | Preservative | Germaben ® IIE (ISP) |
| (and) Diazolidinyl Urea | | | |
| (and) Methylparaben | | | |
| (and) Propylparaben | | | |

NOTES:
*Ingredient 8 was used at a level of 4.5 wt. % of the sum of ingredients 6 and 7.
*Ingredient 9 was used at a level of 6.8 wt. % of the sum of ingredients 6 and 7.
*Ingredient 10 was only used when 2-hydroxy-4-methoxyphenyl)phenylmethanone (BENZO-3) was one of the UV absorbers.

TABLE 2

| Ingredient/ | | | |
|---|---|---|---|
| INCI-CTFA Name | Wt. % | Function | Trade Name (Supplier) |
| PART A | | | |
| 1. Deionized Water | q.s. to 100 | | |
| 2. Carbomer | 0.80 | Rheology | Carbopol ® 980 |
| | | Modifier | polymer (Noveon) |
| 3. C10-30 Alkyl | 0.20 | Rheology | Pemulen ® TR-2 |
| Acrylates | | Modifier | polymer (Noveon) |
| Crosspolymer | | | |
| 4. Propylene Glycol | 2.00 | Humectant | |
| 5. Triethanolamine | 1.00, q.s. to | Neutralizer | |
| (99%) | pH 7.50 | | |
| PART B | | | |
| 6. Deionized Water | 10.00 | | |
| 7. PBSA | See Examples | UV Absorber | |
| 8. Disodium ETDA | 0.10 | Chelating | Versene ™ NA (Dow) |
| | | Agent | |
| 9. Triethanolamine | 1.00, q.s. to | Neutralizer | |
| (99%) | pH 7.50 | | |
| PART C | | | |
| 10. Bis- | See | SPF Booster | Ultrabee ® 25 silicone |
| hydoxyethoxypropyl | Examples | | (Noveon) |
| Dimethicone beeswax | | | |
| esters | | | |
| 11. 2° UV Absorber | See | UV Absorber | |
| | Examples | | |
| 12. Cetearyl Alcohol | See Note | Emulsifier | Promulgen D |
| and Ceteareth-20 | Below | | (Chemron) |
| 13. Stearic Acid | See Note | Emulsifier | Hystrene 5016 NF |
| | Below | | (CK Witco) |
| PART D | | | |
| 14. Triethanolamine | 0.5, q.s. to | Neutralizer | |
| (99%) | pH 7.5 | | |
| PART E | | | |
| 15. Propylene Glycol | 1.00 | Preservative | Germaben ® IIE (ISP) |
| (and) Diazolidinyl Urea | | | |
| (and) Methylparaben | | | |
| (and) Propylparaben | | | |

NOTES:
*Ingredient 12 was used at a level of 4.5 wt. % of the sum of ingredients 10 & 11.
*Ingredient 13 was used at a level of 6.8 wt. % of the sum of ingredients 10 & 11.

### SPF Evaluation

The in vitro SPF values of the sunscreen formulations of Examples 1 to 77 were measured as follows.

A sheet of Vitro-Skin® artificial skin substrate (IMS Inc., Milford, Conn.) was hydrated (placed in a humidifier at 30 to 70% r.h. for 8 to 16 hrs. The hydrated artificial skin substrate was then cut into rectangles measuring 6.2 cm×9.0 cm. The artificial skin substrate rectangles were mounted onto a 6 cm×6 cm GEPE Glassless Slide Mount. Thirty drops of the sunscreen formulation weighing 0.0360-0.0404 grams were dispensed from a 1 ml syringe (26 gauge/⅜″ needle) onto the artificial substrate in an evenly spaced configuration consisting of five columns and six rows. The treated substrate was placed on a plastic covered foam block and the sunscreen was evenly spread into a film by gently rubbing the sunscreen with a cot-covered finger for 30 seconds. The film was allowed to dry for 15 minutes. The analysis of the samples for the determination of SPF values was conducted on a Labsphere UV-1000S Ultraviolet Transmittance Analyzer manufactured by Labsphere, Inc., North Sutton, N.H. The analyzer is manufacturer equipped with Windows compatible application software that automatically calculates the in vitro SPF value of a tested sample. The instrument measures diffuse transmittance of a sample as a function of wavelength in the ultraviolet spectrum at UV wavelengths from 280 nm to 400 nm. The spectral data is processed through an algorithm programmed into the software and is converted to an in vitro SPF value for the tested sample. A detailed explanation of in vitro SPF measurements by the Labsphere UV-1000S Transmittance Analyzer is contained in the technical bulletin entitled SPF Analysis of Sunscreens Using the Labsphere UV-1000S Ultraviolet Transmittance Analyzer, is available as a download from Labsphere, Inc., www.labsphere.com, which is incorporated herein by reference.

TABLE 3
(Examples 1 to 38)

| | | | Wt. % | |
|---|---|---|---|---|
| | | Wt. % UV | Dimethicone | Measured |
| Example No. | UV Absorber | Absorber | Ester Wax | SPF |
| 1 | EHMC | 1.5 | 0 | 1.95 |
| 2 | EHMC | | 1.5 | 2.92 |
| 3 | EHMC | | 2.0 | 2.58 |
| 4 | EHMC | 5.9 | 0 | 5.59 |
| 5 | EHMC | | 1.5 | 8.71 |
| 6 | EHMC | | 2.0 | 6.91 |
| 7 | EHMC | 7.5 | 0 | 6.09 |
| 8 | EHMC | | 1.5 | 7.39 |
| 9 | EHMC | | 2.0 | 10.09 |
| 10 | EHS | 5.0 | 0 | 4.10 |
| 11 | EHS | | 1.5 | 3.64 |
| 12 | EHS | | 2.0 | 3.77 |
| 13 | BENZO-3 | 1.8 | 0 | 3.31 |
| 14 | BENZO-3 | | 1.5 | 3.67 |
| 15 | BENZO-3 | | 2.0 | 5.29 |
| 16 | BENZO-3 | 6.0 | 0 | 4.52 |
| 17 | BENZO-3 | | 1.5 | 3.51 |
| 18 | BENZO-3 | | 2.0 | 2.99 |
| 19 | PBSA | 2.4 | 0 | 7.97 |
| 20 | PBSA | | 1.5 | 7.27 |
| 21 | PBSA | | 2.0 | 8.41 |
| 22 | PBSA | 4.0 | 0 | 8.20 |
| 23 | PBSA | | 1.5 | 9.38 |
| 24 | PBSA | | 2.0 | 8.85 |
| 25 | PADO | 1.5 | 0 | 2.39 |
| 26 | PADO | | 1.5 | 2.83 |
| 27 | PADO | | 2.0 | 2.60 |
| 28 | PADO | 6.2 | 0 | 4.53 |
| 29 | PADO | | 1.5 | 6.79 |
| 30 | PADO | | 2.0 | 7.82 |
| 31 | PADO | 8.0 | 0 | 5.55 |
| 32 | PADO | | 1.5 | 7.08 |
| 33 | PADO | | 2.0 | 7.11 |
| 34 | Oct | 2.5 | 0 | 2.40 |
| 35 | OCTO | | 1.5 | 2.39 |
| 36 | OCTO | | 2.0 | 2.42 |
| 36 | OCTO | 10.0 | 0 | 4.21 |
| 37 | OCTO | | 1.5 | 6.30 |
| 38 | OCTO | | 2.0 | 6.05 |

EHMC = 2-ethylhexyl 4-methoxycinnamate
PBSA = 2-phenylbenzimidazole-5-sulfonic acid
BENZO-3 = 2-hydroxy-4-methoxyphenyl)phenylmethanone
EHS = 2-ethylhexyl salicylate
OCTO = 2-propanoic acid, 2-cyano-3-diphenyl, 2-ethylhexyl ester
PADO = 2-ethylhexyl 4-(dimethylamino)benzoate

TABLE 4
(Examples 39 to 77)

| Exam- | | | | | Wt. % | |
|---|---|---|---|---|---|---|
| ple | 1° UV | Wt. | 2° UV | Wt. | Dimethicone | Measured |
| No. | Absorber | % | Absorber | % | Ester Wax | SPF |
| 39 | BENZO-3 | 6 | EHMC | 0.9 | 0 | 5.93 |
| 40 | BENZO-3 | 6 | EHMC | 0.9 | 1.5 | 7.43 |
| 41 | BENZO-3 | 6 | EHMC | 0.9 | 2 | 6.67 |
| 42 | BENZO-3 | 6 | EHMC | 5.0 | 0 | 12.33 |
| 43 | BENZO-3 | 6 | EHMC | 5.0 | 1.5 | 11.99 |
| 44 | BENZO-3 | 6 | EHMC | 5.0 | 2 | 17.68 |
| 45 | BENZO-3 | 6 | EHMC | 7.5 | 0 | 13.86 |
| 46 | BENZO-3 | 6 | EHMC | 7.5 | 1.5 | 18.62 |
| 47 | BENZO-3 | 6 | EHMC | 7.5 | 2 | 19.24 |
| 48 | PBSA | 4 | EHMC | 4.5 | 0 | 12.83 |
| 49 | PBSA | 4 | EHMC | 4.5 | 1.5 | 13.99 |
| 50 | PBSA | 4 | EHMC | 4.5 | 2 | 15.22 |
| 51 | PBSA | 4 | EHMC | 7.5 | 0 | 16.07 |
| 52 | PBSA | 4 | EHMC | 7.5 | 1.5 | 16.45 |
| 53 | PBSA | 4 | EHMC | 7.5 | 2 | 17.91 |
| 54 | PADO | 8 | EHMC | 6.0 | 0 | 9.02 |
| 55 | PADO | 8 | EHMC | 6.0 | 1.5 | 12.16 |
| 56 | PADO | 8 | EHMC | 6.0 | 2 | 12.59 |
| 57 | PADO | 8 | EHMC | 7.5 | 0 | 11.25 |
| 58 | PADO | 8 | EHMC | 7.5 | 1.5 | 12.59 |
| 59 | PADO | 8 | EHMC | 7.5 | 2 | 10.83 |
| 60 | OCTO | 10 | EHMC | 5.0 | 0 | 10.82 |
| 61 | OCTO | 10 | EHMC | 5.0 | 1.5 | 14.18 |
| 62 | OCTO | 10 | EHMC | 5.0 | 2 | 12.92 |
| 63 | OCTO | 10 | EHMC | 7.5 | 0 | 16.05 |
| 64 | OCTO | 10 | EHMC | 7.5 | 1.5 | 12.03 |
| 65 | OCTO | 10 | EHMC | 7.5 | 2 | 14.48 |
| 66 | EHS | 5 | EHMC | 0.5 | 0 | 4.17 |
| 67 | EHS | 5 | EHMC | 0.5 | 1.5 | 3.84 |
| 68 | EHS | 5 | EHMC | 0.5 | 2 | 9.19 |
| 69 | EHS | 5 | EHMC | 4.8 | 0 | 9.11 |
| 70 | EHS | 5 | EHMC | 4.8 | 1.5 | 5.9 |
| 71 | EHS | 5 | EHMC | 4.8 | 2 | 10.72 |
| 72 | EHS | 5 | EHMC | 7.5 | 0 | 9.67 |
| 73 | EHS | 5 | EHMC | 7.5 | 1.5 | 7.69 |
| 74 | EHS | 5 | EHMC | 7.5 | 2 | 10.12 |
| 75 | EHS | 5 | BENZO-3 | 6 | 0 | 3.72 |
| 76 | EHS | 5 | BENZO-3 | 6 | 1.5 | 9.94 |
| 77 | EHS | 5 | BENZO-3 | 6 | 2 | 11.14 |

EHMC = 2-ethylhexyl 4-methoxycinnamate
PBSA = 2-phenylbenzimidazole-5-sulfonic acid
BENZO-3 = 2-hydroxy-4-methoxyphenyl)phenylmethanone
EHS = 2-ethylhexyl salicylate
OCTO = 2-propanoic acid, 2-cyano-3-diphenyl, 2-ethylhexyl ester
PADO = 2-ethylhexyl 4-(dimethylamino)benzoate

### Examples 78 to 84

The dimethicone copolyol wax of the present invention was post added to selected commercially available sunscreen products that were purchased on the retail market. The initial in vitro SPF values of the commercial unadulterated products were measured on the Labsphere UV-1000S Transmittance Analyzer as set forth in the previous examples and recorded. Following the measurement of the initial (as purchased) in vitro SPF values for each of the commercially available sunscreen products, 2.0 wt. % of Ultrabee® 25 dimethicone copolyol beeswax (Noveon, Inc.) was added (with intimate mixing for 15 minutes) to each product. A final SPF value was measured and recorded. The results are presented in Table 5.

TABLE 5

| Sunscreen | Active | | | | ΔSPF |
|---|---|---|---|---|---|
| Product | Ingredients | Inactive Ingredients | SPFᵢ | SPF_{f} | (%) |
| Coppertone ® | OCTO, | Water, Ethylhexyl Palmitate, Propylene | 7.08 | 7.96 | +12.4 |
| SPF 4 | EHMC | Glycol, Cetyl Phosphate, Acrylates/C10-30 | | | |
| Sunscreen | | Alkyl Acrylate Crosspolymer, | | | |
| Lotion | | Triethanolamine, Tocopherol (vitamin E), | | | |
| | | Aloe Barbadensis Extract, Diazolidinyl Urea, | | | |
| | | Methylparaben, Fragrance, Carbomer, | | | |
| | | Propylparaben, Disodium EDTA | | | |
| Coppertone ® | EHMC, | Water; Sorbitan Sesquioleate; Sorbitol; | 8.57 | 17.19 | +100.6 |
| SPF 8 | BENZO-3 | Glyceryl Stearate SE; Isopropyl Myristate; | | | |
| Sunscreen | | Stearic Acid; Triethanolamine; | | | |
| Lotion | | Octadecene/MA Copolymer; Benzyl Alcohol; | | | |
| | | Fragrance; Dimethicone; Methylparaben; | | | |
| | | Aloe Barbadensis Extract; Jojoba Oil; | | | |
| | | Tocopheryl (Vitamin E); Propylparaben; | | | |
| | | Carbomer; Disodium EDTA | | | |
| Coppertone ® | EHMC, | Water, Sorbitan Oleate, Sorbitol, Glyceryl | 30.88 | 31.39 | +1.7 |
| SPF 15 | BENZO-3 | Stearate SE, Isopropyl Myristate, Stearic | | | |
| Sunscreen | | Acid, Hydrogenated Palm Kernel Oil, | | | |
| Lotion | | Triethanolamine, Octadecene/MA | | | |
| | | Copolymer, Benzyl Alcohol, Fragrance, | | | |
| | | Dimethicone, Methylparaben, Aloe | | | |
| | | Barbadensis Extract, Jojoba Oil, Tocopherol | | | |
| | | (Vitamin E), Imidazolidinyl Urea, | | | |
| | | Propylparaben, Carbomer, Disodium EDTA | | | |
| Banana | BENZO-3 | Acrylates/C10-30 Alkyl Acrylate | 5.90 | 5.51 | −6.6 |
| Boat ® SPF 8 | 1.2%, | Crosspolymer, Aloe Barbadensis Leaf Juice, | | | |
| Sunscreen | PADO 5.4% | Cetyl Alcohol, Cocos Nucifera (Coconut) Oil, | | | |
| Lotion | | Fragrance, Glyceryl Stearate, Hydrolyzed | | | |
| | | Collagen, Imidazolidinyl Urea, Isocetyl | | | |
| | | Alcohol, Methylparaben, Mineral Oil, Mink | | | |
| | | Oil, Polyethylene, Polyglyceryl-3 Dioleate, | | | |
| | | Propylparaben, Quaternium-15, Sorbitol, | | | |
| | | Stearic Acid, Theobroma Cacao (Cocoa) | | | |
| | | Seed Butter, Tocopheryl Acetate, | | | |
| | | Triethanolamine, Water | | | |
| Hawaiian | EHMC, | Not listed. | 8.15 | 10.75 | +31.9 |
| Tropics ® SPF | EHS | | | | |
| 8 Protective | | | | | |
| Tanning | | | | | |
| Lotion with | | | | | |
| Sunscreen | | | | | |
| Hawaiian | EHMC, | Not listed. | 11.97 | 14.53 | +21.4 |
| Tropic ® SPF | EHS, | | | | |
| 10 Carrot | TiO₂ | | | | |
| Tanning | | | | | |
| Lotion with | | | | | |
| Sunscreen | | | | | |

SPFᵢ = Measured SPF (as purchased)
SPF_{f} = Measured SPF (2 wt. % Ultrabee ® 25 Silicone Post-Added)
EHMC = 2-ethylhexyl 4-methoxycinnamate
BENZO-3 = 2-hydroxy-4-methoxyphenyl)phenylmethanone
EHS = 2-ethylhexyl salicylate
OCTO = 2-propanoic acid, 2-cyano-3-diphenyl, 2-ethylhexyl ester
PADO = 2-ethylhexyl 4-(dimethylamino)benzoate

We claim:

1. A sunscreen composition comprising:
a) a dimethicone copolyol wax selected from at least one compound represented by the formula: 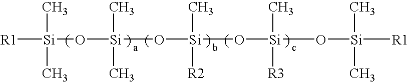
wherein a is an integer ranging from 0 to about 2000; b is an integer ranging from 1 to about 20; and c is an integer ranging from 0 to 20, subject to the proviso that a, b, and c can not all be zero at the same time;
R1 can be the same or different and is —CH₃, or —(CH₂)₃—O—(CH₂CH₂O)ₓ—(CH₂CH(CH₃)O)_{y}—(CH₂CH₂O)ₙ—C(O)R;
R2 is —CH₃, or —(CH₂)₃—O—(CH₂CH₂O)ₓ—(CH₂CH(CH₃)O)_{y}—(CH₂CH₂O)ₙ—C(O)R;
each of x, y, and z in R1 and R2 above is an integer ranging from 0 to 20, with the proviso that R1 and R2 can not be —CH₃ at the same time and x, y, and z can not all be zero at the same time; R3 is —(CH₂)ₙCH₃, with n being an integer from 1 to 17; R is a hydrocarbon group independently selected from about 19 to about 52 carbon atoms; and
b) at least one organic UV absorbing agent selected from 2-ethylhexyl 4-methoxycinnamate, 2-phenylbenzimidazole-5-sulfonic acid, (2-hydroxy-4-methoxyphenyl)phenylmethanone, 2-ethylhexyl salicylate, 2-propanoic acid, 2-cyano-3-diphenyl, 2-ethylhexyl ester, and 2-ethylhexyl 4-(dimethylamino); and
c) a pharmaceutically-acceptable sunscreen carrier.

2. A sunscreen composition of claim 1 wherein x is an integer ranging from 1 to 5 and y and z are 0.

3. A sunscreen composition of claim 1 wherein said dimethicone copolyol wax is selected from a compound of the formula: 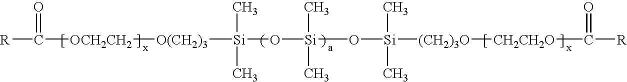
wherein a is an integer from 1 to 2000; x is an integer selected from 1, 2, 3, 5, and 5; and R is a hydrocarbon group independently selected from about 19 to about 52 carbon atoms.

4. A sunscreen composition of claim 1 further comprising at least one inorganic UV absorbing agent selected from zinc oxide, titanium dioxide, calamine, and mixtures thereof.

5. A sunscreen composition of claim 1 wherein said pharmaceutically-acceptable sunscreen carrier is selected from water, natural and synthetic oils, lower alcohols, and mixtures thereof.

6. A sunscreen composition of claim 1 further comprising an additive selected from chelators, fragrances, humectants, lubricants, emollients, neutralizers, preservatives, auxiliary solvents, spreading aids, film forming polymers, viscosity modifiers/emulsifiers, and combinations thereof.

7. A sunscreen composition comprising:
a) from about 0.1 wt. % to about 15 wt. % of a dimethicone copolyol wax selected from at least one compound represented by the formula: 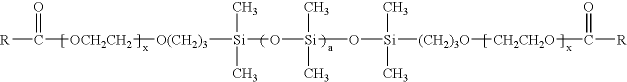
wherein a is an integer from 1 to 2000; x is an integer selected from 1, 2, 3, 4, and 5; and R is a hydrocarbon group independently selected from about 19 to about 52 carbon atoms.
b) from about 0.5 wt. % to about 30 wt. % of at least one organic UV absorbing agent selected from 2-ethylhexyl 4-methoxycinnamate, 2-phenylbenzimidazole-5-sulfonic acid, (2-hydroxy-4-methoxyphenyl)phenylmethanone, 2-ethylhexyl salicylate, 2-propanoic acid, 2-cyano-3-diphenyl, 2-ethylhexyl ester, and 2-ethylhexyl 4-(dimethylamino); and
c) from about 50 wt. % to about 99 wt. % of a pharmaceutically-acceptable sunscreen carrier; and
d) an optional additive selected from chelators, fragrances, humectants, lubricants, emollients, neutralizers, preservatives, auxiliary solvents, spreading aids, film forming polymers, viscosity modifiers/emulsifiers, and combinations thereof.

8. A sunscreen composition of claim 7 wherein said hydrocarbon group is alkyl.

9. A sunscreen composition of claim 7 further comprising at least one inorganic UV absorbing agent selected from zinc oxide, titanium dioxide, calamine, and mixtures thereof.

10. A sunscreen composition of claim 7 wherein said pharmaceutically-acceptable sunscreen carrier is selected from water, natural and synthetic oils, lower alcohols, and mixtures thereof.

11. A method for increasing the SPF value of a sunscreen composition containing an organic UV absorbing agent selected from 2-ethylhexyl 4-methoxycinnamate, 2-phenylbenzimidazole-5-sulfonic acid, (2-hydroxy-4-methoxyphenyl)phenylmethanone, 2-ethylhexyl salicylate, 2-propanoic acid, 2-cyano-3-diphenyl, 2-ethylhexyl ester, and 2-ethylhexyl 4-(dimethylamino) by adding an effective amount of at least one dimethicone copolyol wax compound represented by the formula: 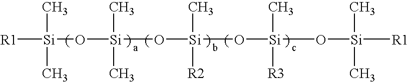
wherein a is an integer ranging from 0 to about 2000; b is an integer ranging from 1 to about 20; and c is an integer ranging from 0 to 20, subject to the proviso that a, b, and c can not all be zero at the same time;
R1 can be the same or different and is —CH₃, or —(CH₂)₃—O—(CH₂CH₂O)ₓ—(CH₂CH(CH₃)O)_{y}—(CH₂CH₂O)ₙ—C(O)R;
R2 is —CH₃, or —(CH₂)₃—O—(CH₂CH₂O)ₓ—(CH₂CH(CH₃)O)_{y}—(CH₂CH₂O)ₙ—C(O)R;
each of x, y, and z in R1 and R2 above is an integer ranging from 0 to 20, with the proviso that R1 and R2 can not be —CH₃ at the same time and x, y, and z can not all be zero at the same time; R3 is —(CH₂)ₙCH₃, with n being an integer from 1 to 17; and R is a hydrocarbon group independently selected from about 19 to about 52 carbon atoms.

12. A method of claim 11 wherein x in said copolyol wax formula is an integer from 1 to 5 and y and z are 0.

13. A method of claim 11 wherein said dimethicone copolyol wax is selected from a compound of the formula: 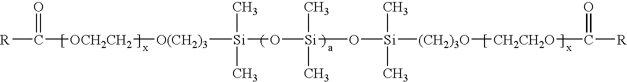
wherein a is an integer from 1 to 2000; x is an integer selected from 1, 2, 3, 4, and 5; and R is a hydrocarbon group independently selected from about 19 to about 52 carbon atoms.

14. A method of claim 13 wherein said hydrocarbon group is alkyl.
